(19) United States
(12) Patent Application Publication
(10) Pub. No.: US 2008/0215102 A1
(43) Pub. Date: Sep. 4, 2008

(54) Method and system aiding decision making during CPR

(75) Inventors: Helge Myklebust, Stavanger (NO); Joar Eilevstjonn, Sandnes (NO)

Correspondence Address: Karen Lenaburg, Esq.;DORSEY & WHITNEY LLP, Suite 3400, 1420 Fifth Avenue, Seattle, WA, 98101, US

(73) Assignee: Laerdal Medical AS, Stavanger (NO)

(21) Appl. No.: 12/070,916

(22) Filed: Feb. 20, 2008

(30) Foreign Application Priority Data
Feb. 20, 2007 (GB) 0703259.2

(51) Int. Cl. A61N 1/00

(52) U.S. Cl. 607/6; 607/5

(57) ABSTRACT

An embodiment of the invention is a method for recommending actions to be taken during resuscitation of a patient. Input signals related to the resuscitation are received during the resuscitation. The input signals are processed to generate output signals based on the input signals and predetermined criteria. The output signals are representative of the actions to be taken and are provided for further action.

### CROSS-REFERENCE TO RELATED APPLICATIONS

This application claims the benefit of United Kingdoms Patent Application No. 0703259.2, filed on Feb. 20, 2007, under 35 U.S.C. 119. The entire disclosure of the prior application is considered to be part of the disclosure of the instant application and is hereby incorporated by reference therein.

### TECHNICAL FIELD

Embodiments of the invention relate to cardiopulmonary resuscitation (CPR) and, more particularly, to method and system aiding decision making during a CPR process.

### BACKGROUND OF THE INVENTION

Presently, the Guidelines for CPR in principle advocate a single-treatment recommendation for all patients, although deviation from the recommended treatment is allowed. For example, a medical director might advocate that some CPR should be carried out before defibrillation for ventricular fibrillation (VF) arrests. As another example, some rescuers might be unwilling to perform mouth to mouth ventilation. While recent studies have demonstrated that one alternative treatment benefits patients of VF arrest, the alternative treatment does not necessarily benefit patients showing non-VF rhythms. For instance, some groups of patients (e.g., asystole patients) might benefit from certain drugs or drug combinations. On the other hand, gasping has been shown to be associated with improved hemodynamics at least for some other patients.

All in all, there is evidence that some patients might benefit from a custom-tailored therapy rather than a one-size-fits-all treatment currently advocated. More specifically, it would be beneficial that the sequence, timing, duration, and/or dose of the available therapy elements, including compressions, ventilations, defibrillation and medication, could be adjusted for each patient and determined on a case-by-case basis.

United States Patent Publication No. 2005/0197672 A1 describes a resuscitation system, which comprises devices for delivering defibrillation shocks, and devices for detecting an electrocardiogram (ECG) signal to determine whether the cardiac rhythm is shockable or not. This determination is based on detected ECG signal during periods where CPR chest compression is not delivered. This is due to the fact that CPR produces artifacts in the ECG signal, which cannot be used to assess the heart's state. A typical detection time sequence for this device comprises a fixed compression time followed by a pause during which the heart state is assessed and defibrillation (if necessary) is performed.

In other devices, a process that alternately delivers defibrillation and CPR according to a fixed pattern is carried out. With such devices, an analysis of the patient's condition is performed after a predetermined time has elapsed, and outcome of the analysis is used to decide how to proceed further. Still, because the analysis is performed after the predetermined time has elapsed, it is not performed continuously throughout the resuscitation of the patient.

There is, therefore, a need for a method and system for decision-making support during cardiac arrest resuscitation to continuously provide recommendation regarding actions to be taken in order to minimize required pauses in the treatment.

### BRIEF DESCRIPTION OF THE DRAWINGS

FIG. 1 is a general block diagram of a system for aiding decision making during CPR in accordance with an embodiment of the invention.

FIG. 2 is a diagram showing a process performed by the system of FIG. 1 in accordance with an embodiment of the invention.

FIG. 3 is a timing diagram showing segments of an ECG waveform for analysis in accordance with an embodiment of the invention.

### DETAILED DESCRIPTION OF PREFERRED EMBODIMENTS

Certain details are set forth below to provide a sufficient understanding of embodiments of the invention. However, it will be clear to one skilled in the art that embodiments of the invention may be practiced without these particular details. Moreover, the particular embodiments of the present invention described herein are provided by way of example and should not be used to limit the scope of the invention to these particular embodiments.

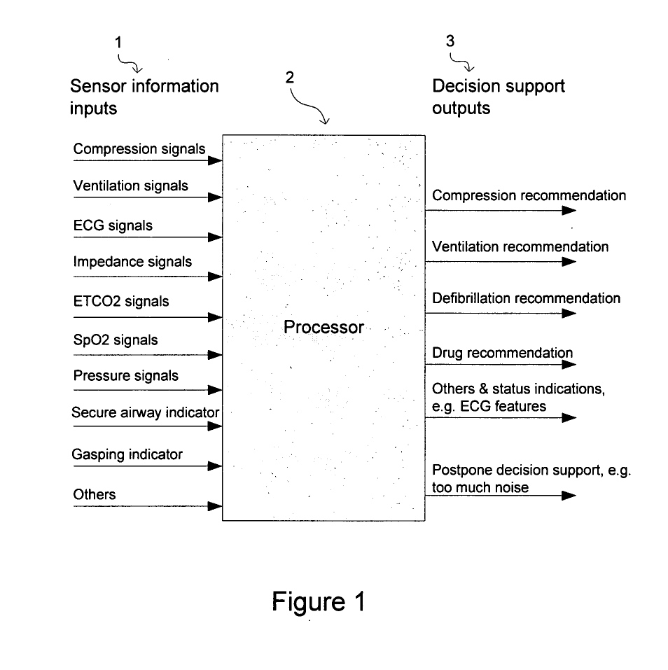

FIG. 1 illustrates a general block diagram a system, including input and output signals, for aiding decision making during CPR in accordance with an embodiment of the invention. In general, the system receives inputs signals 1 from which information related to the status of the resuscitation process (that is, the status of the therapy and the condition of the patient) may be derived. A processor 2 processes the information and provides outputs 3 that represent recommendations as to what actions should be taken.

Sensors (not shown) for receiving the input signals 1 may include sensors for compression, ventilation, impedance, electrocardiogram (ECG), end-tidal carbon dioxide (ETCO₂) and pressure, as well as indicators/detectors for a secure airway. Decision support, in terms of recommendations, is provided in the form of output signals 3. These output signals 3 include recommendations on, for example, compressions, ventilations, defibrillation, drugs, and characteristics of the process, etc. Possible receivers of the output signals 3 include, for example, AEDs (automatic external defibrillators), ALS-monitors/defibrillators, mechanical chest compression machines, ventilation/respiration monitors, or various combinations of such devices. A rescuer will in general receive decision support from one of the aforementioned devices in some embodiments, but it is also possible for the system to provide decision support directly to the rescuer in other embodiments of the invention. The output signals 3 may also be used to automatically control actions in a receiving device or to provide information allowing the user to make the decision with respect to next-step actions. In one embodiment, the processor 2 may be a processor of a device that utilizes the output signals 3. In another embodiment, the processor 2 may be part of a stand-alone device that provides the output signals 3 to other devices that utilize the output signals.

A method according to the system just described may comprise the step of receiving by a input unit the input signals 1 related to the resuscitation process, such as, for example, compressions (e.g., parameterized information or signals correlated with compressions, such as depth, force, and acceleration), ventilations, ECG, impedance, ETCO₂, SpO₂, pressure, and secure airway indication. Some of the aforementioned signals may be related to the patient's bodily functions (e.g., ECG, impedance, ETCO₂, SpO₂, pressure) while others may be related to the resuscitation process (e.g., compressions, ventilations, secure airway indication). The input unit may be connected to a resuscitation machine and it can also receive inputs directly from sensors on the patient.

The method also includes the step of processing the input signals 1 by the processor 2 to provide a representation of the resuscitation process based on the input signals 1 and predetermined criteria. The representation is used to generate output signals 3 representative of actions to be taken during resuscitation and/or characteristics of the process (e.g., VF vitality). The predetermined criteria may be, for example, those criteria established in the International Guidelines for CPR. Of course, the predetermined criteria need not be limited to the context of the International Guidelines for CPR and may comprise other predetermined criteria, as will be appreciated by those ordinarily skilled in the art. Processing of the input signals 1 by the processor 2 may comprise filtering of ECG signals and analysis of parameters of the filtered signals to check for onset of a shockable rhythm. The method further includes the step of delivering the output signals 3. As mentioned above, output signals 3 may be delivered to resuscitation machines, defibrillator or other type of client devices, or to one or more rescue personnel.

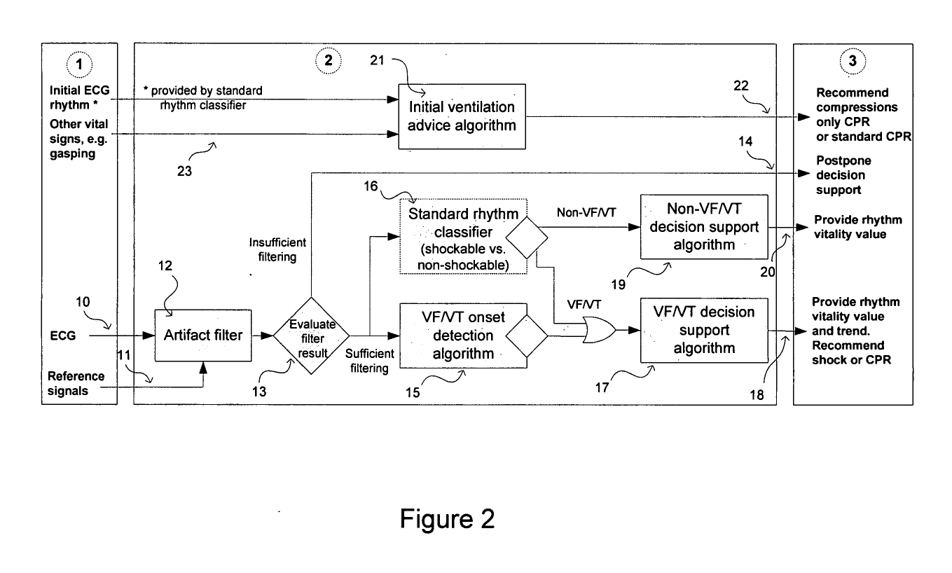

FIG. 2 shows a process performed by the system of FIG. 1 in accordance with an embodiment of the invention. In one embodiment, the process of FIG. 2 may be used in conjunction with a defibrillator. In another embodiment, the process of FIG. 2 may be utilized as part of the operation of a defibrillator. Elements of the process shown in FIG. 2 are grouped into three groups each with a number circled in dotted line corresponding to the numeral reference of FIG. 1. More specifically, elements in the first group comprise input signals 1, elements in the second group comprise the processing steps performed by the processor 2, and elements of the third group comprise output signals 3.

In one embodiment of the invention, the process performed by the processor 2 includes filtering of ECG signals to remove CPR noise. This step allows the possibility of monitoring ECG during most of the resuscitation process. It is thus no longer necessary to interrupt the CPR in order to obtain ECG signals to decide whether defibrillation is necessary or not. Accordingly, ECG signals can be monitored continuously and CPR will only be interrupted when defibrillation is actually taking place. If the filtered ECG signal does not denote that defibrillation is necessary, the CPR process may continue uninterrupted. In an embodiment, a system may also include an evaluation algorithm to detect when filtering is sufficient (that is, when the results of the filtering can be trusted), including periods of time when compressions are being performed.

According to an embodiment of the invention, ECG signals are processed to detect the onset of a shockable rhythm indicative of ventricular fibrillation (VF) or pulseless ventricular tachycardia (VT). In the rest of the specification, VF, VT and the term shockable rhythm are used interchangeably. Ventricular fibrillation typically deteriorates over time and the CPR provided may not be sufficient to stop or reverse it. In general, the probability of ROSC (Restoration of Spontaneous Circulation) shock seems to be highest near onset of ventricular fibrillation (e.g., within <30 seconds). Therefore, it is important to have the ability to detect a strong ventricular fibrillation at its onset. AEDs typically have an algorithm for distinguishing a shockable rhythm from a non-shockable rhythm. However, the algorithm might be computationally heavy, seldom run continuously, and not giving reliable results in the presence of compressions. The aim of this embodiment of the invention is to provide a method for detecting ventricular fibrillation onset continuously, and during chest compressions, by using artifact filtering.

Detection of a shockable rhythm will, in one embodiment, trigger monitoring of VF vitality (reflecting the state of the myocardium) and trending of VF vitality to assess whether defibrillation shock or CPR should be recommended. VF vitality in the context of the present application refers to a value or values derived from the ECG signals. These values may be, for example, the mean or median slope of the ECG signals. Other values may be median frequency, amplitude, etc. Slope deviation can be used to assess vitality/quality of a non-shockable rhythm. Accordingly, one method for detection of ventricular fibrillation onset according to the invention comprises calculation of mean slope, median slope, slope deviation and rate for CPR-filtered ECG signals. It is also possible to calculate such parameters for non-filtered signals, especially in the case where CPR is not being performed.

As mentioned before, prior art devices are able to detect the presence of ventricular fibrillation at a determined point in time. They are, however, not made to detect onset of ventricular fibrillation and will work poorly during ongoing chest compressions. In contrast, continuous detection combined with appropriate processing of determined characteristics of the ECG signals and their variation, according to an embodiment of the invention, will provide an indication of VF onset.

As shown in FIG. 2, in one embodiment, ECG signals 10 are first processed by the a artifact filter 12. As mentioned previously, chest compressions during CPR will typically cause artifacts in the ECG—often inhibiting further ECG analysis. To remove or reduce these artifacts in order to enable subsequent ECG analysis, a filter such as the adaptive filter 12 may be used. An example of such a filter may be the MultiChannel Robust Adaptive Matching Pursuit (MC-RAMP) filter as described in J. H. Husoy, J. Eilevstjonn, T. Eftestol, S. O. Aase, H. Myklebust, and P. A. Steen, “Removal of cardiopulmonary resuscitation artifacts from human ECG using an efficient matching pursuit-like algorithm,” IEEE Trans Biomed Eng, vol. 49, pp. 1287-98, November 2002. Alternatively, another example of such a filter may be the one disclosed in J. Eilevstjonn, T. Eftestol, S. O. Aase, H. Myklebust, J. H. Husoy, and P. A. Steen, “Feasibility of shock advice analysis during CPR through removal of CPR artifacts from the human ECG,” Resuscitation, vol. 61, pp. 131-41, May 2004. Use of the adaptive filter 12 requires at least one reference channel 11 providing an artifact correlated signal. Typically, reference signals such as compression acceleration, depth, force, thorax impedance and ECG common mode voltage can be provided by using a sensor between the hand of the rescuer and the victim's chest in addition to defibrillation pads. Although primarily adapted for filtering chest compression artifacts, the artifact filter 12 may also be capable of filtering noise generated by, for example, ventilations, electrical discharges and any other noise present in reference channels 11.

In the embodiment illustrated in FIG. 2, the system determines whether or not the ECG signals 10 is filtered satisfactorily in step 13, filter result evaluation, after detection of compressions (e.g., using signals/energy provided in one of the reference channels 11). Results of this evaluation may be presented as a “continuous” quality measure or as a boolean value (trust/do not trust or good/bad). The following are examples of some of the situations where the evaluation may report an unsatisfactory filtering.

1) Very high amplitude in the ECG signal prior to the artifact filtering (e.g., >1.5 mV). The algorithm used by the processor 2 may have a threshold on the number of high amplitude peaks present and/or on portion of high amplitude samples in a segment—correlated and/or non-correlated to the chest compressions. The amplitude threshold might differ depending on whether the high amplitude peaks are correlated with the chest compressions or not.

2) Very high compression rates (e.g., >150 per minute). High compression rates might cause artifacts resembling a shockable rhythm and might be problematic if artifact filtering only removes part of the artefacts.

3) Chest compression rate similar to the intrinsic rate of an organized rhythm (e.g., Pulseless Electrical Activity, or PEA). Artifact filtering may in this case remove too much of the underlying heart rhythm and thus should not be used, or alternatively it should be adjusted to accommodate this situation.

4) Shape (e.g., sharpness/spikiness of compression peak) of the probable ECG compression artifacts differs substantially from the shape of the compressions as presented in the reference channels 11. That is, there might be situations where it is likely that the reference channels 11 do not contain enough information about the artifacts in the ECG signals (e.g., due to nonlinearities).

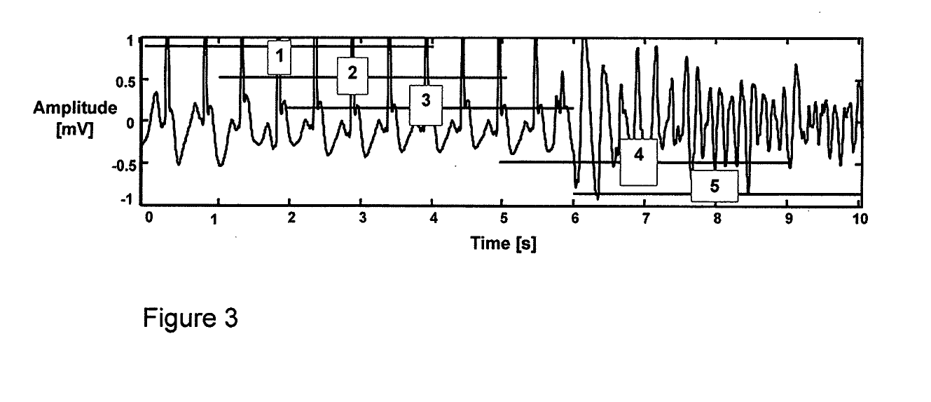

If the artifact filtering is not considered satisfactory by filter result evaluation in step 13 (e.g., insufficient filtering), the process proceeds to output a signal 14 indicating that decision support cannot be given at the present moment. If, however, the artifact filtering is considered satisfactory (e.g., sufficient filtering), the process continues in steps 15 and 16 where VF/VT onset detection and standard rhythm classification (e.g., shockable versus non-shockable rhythms), respectively, are performed. It should be noted that step 16 may be optional and not necessary to practice embodiments of the invention. In other words, certain embodiments of the invention may detect only VF/VT onset but not perform standard rhythm classification. For each iteration/sample, the input provided for processing in step 15 may be a segment of artefact-filtered ECG waveform (e.g., in segments of 10-second length). In step 15, changes in features of the ECG waveform are calculated and monitored for sub-segments of each of the segments. FIG. 3 shows an example of such segmentation. In the embodiment of the invention shown, there are five partly overlapping sub-segments, each of 4-second length. As shown in FIG. 3, sub-segment 1 starts at time 0, sub-segment 2 at time 1, sub-segment 3 at time 2, sub-segment 4 at time 5, and sub-segment 5 at time 6. The increased interval between start times of sub-segments 3 and 4 is because features from sub-segments 1-3 represent feature values prior to a potential VF onset while sub-segments 4 and 5 represent values after a potential VF onset. The length and the distribution of the segments may be predefined, and the sequence of the segments (1-2-3-4-5) may be sampled continuously in one embodiment. Alternatively, other embodiments of the invention may use other sequences and lengths of segments.

The ECG features used in this embodiment of the invention for detecting VF onset include: mean slope (first derivative) of the ECG waveform, median slope (first derivative) of the ECG waveform, slope deviation (relative difference between mean and median slope, as expressed by the equation [mean slope−median slope]/median slope), rate (as expressed by the equation waveform frequency/rate, and may be QRS complex rate where QRS is an electrocardiographic complex consisting of the Q, R, and S waves that represent propagation of a wave of depolarization over the ventricles and commonly measured in beats per minute, or bpm). These features will be defined below:

Mean slope is the mean of the absolute values of the first derivative of the ECG waveform in a segment, e.g. calculated as

$$mean\_slope = \frac{{f}_{s}}{N} {\sum }_{n = 1}^{L} \vert x ( n ) - x ( n - 1 ) \vert$$

where x(n) is an ECG sample in a segment of length L, and fs is the sampling rate.

Median slope is the median of the absolute values of the first derivative of the ECG waveform in a segment, e.g. calculated as

$$median\_slope = {f}_{s} {median}_{n} \vert x ( n ) - x ( n - 1 ) \vert$$

where x(n) is an ECG sample in a segment of length L, and fs is the sampling rate.

Slope deviation is the relative difference between mean and median slope, defined as

$$slope\_deviation = \frac{mean\_slope - median\_slope}{median\_slope}$$

The ECG waveform rate is the most “dominant” frequency of the ECG rhythm, such as rate of QRS complexes or beats in an organized rhythm or frequency of dominant VF waveform. The rate can be calculated in many ways For example, it may be calculated using autocorrelation, counting zero crossings or local maxima/minima (spikes), etc.

VF onset detection in step 15 is based on the fact that the ECG feature slope deviation typically has a high value during a non-shockable rhythm while it is typically low for shockable rhythms. To report the onset of a strong VF, the algorithm checks and requires at least one of the following conditions to be true (using typical threshold values):

1) Clear transition to VF: Median slope is larger than 4 mV/s in last two sub-segments (4 & 5) (only strong VFs are to be detected), slope deviation in last two sub-segments (4 & 5) are below a threshold value of 0.5, slope deviation in at least two of the first three sub-segments (1-3) is above a threshold value 0.5, maximum slope deviation value in sub-segments 1-3 is above (or equal to) a higher threshold, e.g., 0.75, and rate of sub-segments 4 and 5 are all above 190 bpm with one of them also above 200 bpm.

2) More subtle transition to VF: Median slope is larger than 4 mV/s in last two sub-segments (4 & 5) (only strong VFs are to be detected), slope deviation in last two sub-segments (4 & 5) are below a threshold value of 0.5, slope deviation in at least two of the first three sub-segments (1-3) are above a threshold value 0.5, maximum slope deviation value in sub-segments 1-3 is below a higher threshold, e.g. 0.75, rate of sub-segments 4 and 5 are all above 190 bpm with one of them also above 200 bpm, rate of sub-segments 1-3 are all below 190 bpm, and the average rate of sub-segments 1-3 is 30 bpm below the average rate of sub-segments 4-5.

Transition to VF from asystole: Median slope is larger than 4 mV/s in last two sub-segments (4 & 5) (only strong VFs are to be detected), slope deviation in last two sub-segments (4 & 5) are below a threshold value of 0.5, maximum median slope value in sub-segments 1-2 is below 0.5 mV/s (indicating asystole), median slope value in sub-segment 3 is below 1 mV/s, and rate of sub-segments 4 and 5 are all above 190 bpm with one of them also above 200 bpm.

Other criteria for VF onset which differ from the aforementioned rules can be used in other embodiments of the invention. Upon detection of a strong VF onset in any of the above-mentioned alternatives in step 15, the process proceeds to step 17. In step 16, a rhythm classifier detects whether or not there is VF, for example, in a standard fashion as known in the art. If VF is detected the process proceeds to step 17.

If VF is present at the start of treatment (initial VF) or after later detection of VF when the patient was not immediately shocked, the process will proceed to step 17, where the vitality of the VF is analyzed and trended by a VF/VT decision support algorithm. In one embodiment of the invention, median or mean slope is used as a measure of VF vitality, where the VF vitality is sampled at fixed time intervals. The trend of VF vitality samples may be estimated using linear regression estimation in a least-squares sense, for example. The trending process may use several samples (e.g., samples from 30 or 60 seconds of time) to report significant positive or negative trends. Vitality and trend values are compared to thresholds and evaluated further to provide recommendations. An example of the tests that are used to control this process may be as follows: 1) If non-shockable rhythm or a time less than 15 seconds has past since start of the trend analysis, recommend CPR; 2) recommend shock after 5 seconds with flat and/or negative trend after a previous positive trend for a period of at least 5 seconds; 3) recommend shock after 180 seconds with flat trend where there is no previous positive trend of a period of at least 5 seconds; 4) recommend shock after 5 seconds with negative trend; or 5) otherwise, recommend CPR. These tests may include those predetermined criteria established by CPR guidelines, as known in the art, and may also include customized rules.

After the VF/VT decision support algorithm applies the aforementioned tests, the process proceeds to provide output signals via output channel 18 for recommendation. These output signals may be provided to one or more output units, such as the output unit 3 of FIG. 1. The output unit 3 will then communicate these output signals by means of, for example, a display showing an icon, a voice prompt, or control signals to other client devices. It is also possible to output a value or values representing VF vitality, such as a curve, numerical values, a column display, etc.

In case that the process of FIG. 2 detects non-VF/VT rhythms, the process proceeds to step 19. In step 19, a rhythm vitality value is evaluated by a non-VF/VT decision support algorithm. Various non-shockable rhythms can be said to reflect different vitalities of the heart—including probability of return of spontaneous circulation—from asystole to broad-complex “poor” PEAs to narrow-complex PEAs and pulse rhythms. For the purpose of quantifying such rhythms, the ECG feature slope deviation can be used as an indicator—possibly in conjunction with a feature such as mean or median slope and rate (beats per minute). PEAs associated with higher likelihood of leading to a pulse rhythm (or perhaps converting to a strong VF) tend to have higher values of slopes deviation and/or mean slope. Conversely, poor PEAs and asystole tend to have low values. Afterwards, the process provides output signals via output channel 20, and the output signals will comprise characteristics of the rhythm.

In step 21 of the process, the necessity of ventilation is evaluated by an initial ventilation advice algorithm. Before a secured airway is provided (e.g., by means of an endotracheal tube), chest compressions must be interrupted for each ventilation. However, this typically interrupts the blood flow generated by the chest compressions and is thus detrimental to the patient. Studies have shown that, at least for certain group of patients, it may be beneficial to omit ventilations and perform compressions only. One situation where this procedure may be applicable is during witnessed VF arrest. Based on this criterion, the decision support system will in step 21 decide if ventilations are necessary using the following rules (criteria): If it is an initial VF, then postpone start of ventilations until VF is terminated or after a given time (e.g., 5 minutes); but for other initial rhythms, perform ventilations according to current guidelines. Based on the available sensor information, other rules may also be applied. For example, other rules may include the following: 1) If present rhythm is VF, withhold ventilations until VF is terminated or after a given time (e.g., 5 minutes); 2) if present rhythm is PEA, deliver ventilations as per current guidelines; 3) if present rhythm is asystole, deliver ventilations as per current guidelines and also give a drug suitable for asystole patients; or 4) if spontaneous gasping (step 23) is present, withhold ventilations—otherwise deliver ventilations as per current guidelines. The algorithm in step 21 will output a signal 22 recommending compressions only or standard (i.e. compressions and ventilations) CPR. This part of the process may be omitted in alternative embodiments of the invention.

As those ordinarily skilled in the art will appreciate, embodiments of the invention provide the possibility of monitoring and analyzing a resuscitation process while it is being performed. From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for recommending actions to be taken during resuscitation of a patient, comprising:
receiving input signals related to the resuscitation during the resuscitation;
processing the input signals to generate output signals representative of the actions to be taken, the output signals generated based on the input signals and predetermined criteria; and
providing the output signals.

2. The method of claim 1 wherein the input signals comprise at least one of compression signals, ventilation signals, electrocardiogram (ECG) signals, impedance signals, end-tidal carbon dioxide (ETCO₂) signals, saturation of peripheral oxygen (SpO₂) signals, pressure signals and secure airway indications.

3. The method of claim 2 wherein processing the input signals comprises filtering ECG signals to remove CPR noise.

4. The method of claim 3 wherein processing the input signals further comprises MultiChannel Robust Adaptive Matching Pursuit (MC-RAMP) filtering of the ECG signals using at least one of the compression signals, ventilation signals, impedance signals, ETCO₂ signals, SpO₂ signals, pressure signals and secure airway indications as reference, and wherein compression signals comprise signals representative of depth, acceleration and force.

5. The method of claim 1 wherein processing the input signals comprises processing of ECG signals to detect onset of a shockable rhythm indicative of ventricular fibrillation (VF) or pulseless ventricular tachycardia (VT).

6. The method of claim 5 wherein processing the input signals further comprises calculating mean slope, median slope, slope deviation and rate for ECG signals.

7. The method of claim 1 wherein the actions to be taken comprise at least one of providing compressions, ventilations, defibrillating, providing medication or a combination thereof.

8. The method of claim 1 wherein providing the output signals comprises providing the output signals to at least one of an operation control unit of a defibrillator, a patient monitor, a ventilation machine, a CPR machine, a CPR assist/guidance device, a drug delivery machine and a rescuer.

9. A method for deciding actions to be taken regarding cardiopulmonary resuscitation (CPR) of a patient, comprising:
monitoring an electrocardiogram (ECG) signal and at least one reference signal related to the CPR as the CPR is performed on the patient;
filtering the ECG signal using the at least one reference signal to provide a filtered ECG signal; and
detecting onset of a shockable rhythm indicative of ventricular fibrillation (VF) or pulseless ventricular tachycardia (VT) using the filtered ECG signal.

10. The method of claim 9, further comprising:
determining whether or not filtering of the ECG signal is sufficient after the ECG signal has been filtered using the at least one reference signal;
providing a signal representative of postponement of provision of recommendation when filtering of the ECG signal is determined to be insufficient; and
analyzing the filtered ECG signal to provide at least rhythm vitality value when filtering of the ECG signal is determined to be sufficient.

11. The method of claim 10 wherein analyzing the filtered ECG signal comprises analyzing and trending vitality of VF when onset of VF is detected to provide output signals indicative of at least one of providing cardiac rhythm vitality value and trend and recommendation for defibrillation or continuation of the CPR.

12. The method of claim 9, further comprising evaluating necessity of ventilation based on initial ECG rhythm and other vital signs of the patient to provide output signals indicative of at least one of providing cardiac rhythm vitality value and recommendation for compressions-only CPR or standard CPR.

13. A system for determining actions to be taken during resuscitation of a patient, comprising:
an input unit operable to receive input signals related to the resuscitation;
a processing unit coupled to the input unit and operable to process the input signals to generate a representation of the resuscitation, the processing unit further operable to compare the representation with predetermined criteria to generate output signals representative of the actions to be taken; and
an output unit coupled to the processing unit and operable to provide the output signals.

14. The system of claim 13 wherein the input unit is connected to at least one of compression sensors, ventilation sensors, electrocardiogram (ECG) devices, impedance sensors, end-tidal carbon dioxide (ETCO₂) sensors, saturation of peripheral oxygen (SpO₂) sensors, pressure sensors and secure airway indicators.

15. The system of claim 14 wherein the processing unit comprises devices operable to filter ECG signals to remove CPR noise.

16. The system of claim 15 wherein the devices are operable to perform MultiChannel Robust Adaptive Matching Pursuit (MC-RAMP) filtering of the ECG signals using at least one of the compression signals, ventilation signals, impedance signals, ETCO₂ signals, SpO₂ signals, pressure signals and secure airway indications as reference, and wherein compression signals comprise signals representative of depth, acceleration and force.

17. The system of claim 13 wherein the processing unit comprises devices operable to process ECG signals to detect onset of a shockable rhythm indicative of ventricular fibrillation (VF) or pulseless ventricular tachycardia (VT).

18. The system of claim 17 wherein the devices are operable to calculate mean slope, median slope, slope deviation and rate for ECG signals.

19. The system of claim 13 wherein the output unit provides indication of at least one of providing compressions, ventilations, defibrillating, providing medication or a combination thereof.

20. The system of claim 13 wherein the output signals are provided to at least one of an operation control unit in a defibrillator, a patient monitor, a ventilation machine, a CPR machine, a CPR assist/guidance device, a drug delivery machine and a rescuer.

21. The system of claim 13 wherein the system comprises a defibrillator, and wherein the output signals are used to control operation of the defibrillator.

22. A system for aiding decision making during cardiopulmonary resuscitation (CPR) of a patient, comprising:
an input unit operable to receive input signals related to the CPR while the CPR is carried out;
a processing unit coupled to the input unit, the processing unit operable to filter the input signals and generate output signals indicative of at least cardiac rhythm vitality value when filtering of the input signals is satisfactory; and
an output unit coupled to the processing unit and operable to provide the output signals.

23. The system of claim 22 wherein the processing unit is further operable to detect onset of a shockable rhythm indicative of ventricular fibrillation (VF) or pulseless ventricular tachycardia (VT) and generate output signals recommending defibrillation or continuation of the CPR.

24. The system of claim 22 wherein the processing unit is further operable to evaluate necessity of ventilation based on initial electrocardiogram (ECG) rhythm and other vital signs of the patient and generate output signals recommending actions to be taken, and wherein the actions to be taken comprise providing cardiac rhythm vitality value and recommending compressions-only CPR or standard CPR.

25. The system of claim 22 wherein the input signals comprise an ECG waveform of the patient and at least one reference signal related to the CPR, and wherein the processing unit is operable to analyze a plurality of overlapping segments of the ECG waveform in a select one of a plurality of sequences, each segment of the ECG waveform having a variable length depending on the select sequence.
